(19) United States
(12) Patent Application Publication
(10) Pub. No.: US 2012/0101168 A1
(43) Pub. Date: Apr. 26, 2012

(54) DEUTERIUM ENRICHED RASAGILINE

(76) Inventors: Eliezer Bahar, Tel Aviv (IL); Anton Frenkel, Netanya (IL); Victor Piryatinsky, Netanya (IL)

(21) Appl. No.: 13/281,054

(22) Filed: Oct. 25, 2011

Related U.S. Application Data
(60) Provisional application No. 61/406,740, filed on Oct. 26, 2010.

(51) Int. Cl. A61K 31/135; C07C 309/04; C07C 209/14; A61P 25/28; A61P 25/16; C07C 211/42; C07C 309/29

(52) U.S. Cl. 514/657; 564/428; 562/590; 562/114; 562/595; 562/607; 562/493; 562/84

(57) ABSTRACT

The subject invention provides deuterated rasagiline, its salts and uses.

This application claims the benefit of U.S. Provisional Application No. 61/406,740, filed Oct. 26, 2010, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

U.S. Pat. Nos. 5,532,415, 5,387,612, 5,453,446, 5,457,133, 5,599,991, 5,744,500, 5,891,923, 5,668,181, 5,576,353, 5,519,061, 5,786,390, 6,316,504, 6,630,514 disclose R(+)-N-propargyl-1-aminoindan (“R-PAI”), also known as rasagiline, and its uses. Rasagiline mesylate in a 1 mg tablet is commercially available for the treatment of idiopathic Parkinson's disease as AZILECT® from Teva Pharmaceuticals Industries Ltd. (Petach Tikva, Israel) and H. Lundbeck A/S (Copenhagen, Denmark).

### SUMMARY OF THE INVENTION

The subject invention provides a deuterium enriched compound having the structure:

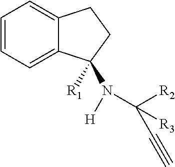

or a pharmaceutically acceptable salt thereof, wherein R₁-R₃ are independently H or D, and wherein at least one of R₁-R₃ is deuterium enriched.

The subject invention also provides a pharmaceutical composition comprising the deuterium enriched compound described herein, or a pharmaceutically acceptable salt thereof, and a pharmaceutically acceptable carrier.

The subject invention further provides a mixture of at least two different deuterium enriched compounds, each compound having the structure:

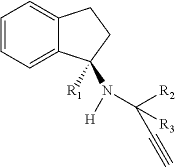

or pharmaceutically acceptable salts thereof, wherein R₁-R₃ are independently H or deuterium enriched.

The subject invention yet further provides a pharmaceutical composition comprising the mixture described herein, or a pharmaceutically acceptable salt thereof, and a pharmaceutically acceptable carrier.

The subject invention yet further provides a method of treating a neurodegenerative disorder in a subject in need thereof, the method comprising periodically administering to the subject in need a therapeutically effective amount of a dosage form comprising as an active ingredient the deuterium enriched compound described herein, or a pharmaceutically acceptable salt thereof, thereby to effectively treat the subject.

The subject invention yet further provides a method of reducing the rate of progression of Parkinson's disease in an early stage Parkinson's disease patient, the method comprising periodically administering to an early stage Parkinson's disease patient an amount of the deuterium enriched compound described herein, or a pharmaceutically acceptable salt thereof, effective to reduce the rate of progression of Parkinson's disease of the early stage Parkinson's disease patient.

The subject invention yet further provides a process for the preparation of a deuterium enriched compound having the structure:

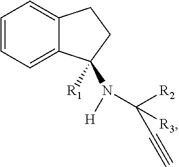

wherein R₁ is D and R₂ and R₃ are independently H or D, the process comprising:
a) reacting

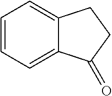

with LiAlD4 in the presence of a solvent to obtain

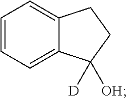

b) converting

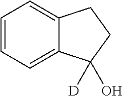

to obtain racemic N-propargyl aminoindan; and
c) separating the racemic N-propargyl aminoindan using a chiral separation method to obtain the compound.

### DETAILED DESCRIPTION OF THE INVENTION

The subject invention provides a deuterium enriched compound having the structure:

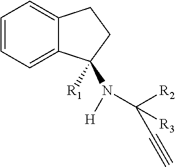

or a pharmaceutically acceptable salt thereof, wherein R₁-R₃ are independently H or D, and wherein at least one of R₁-R₃ is deuterium enriched.

In an embodiment of the deuterium enriched compound or a pharmaceutically acceptable salt thereof, R₁ is deuterium enriched, and each of R₂ and R₃ is H.

In another embodiment of the deuterium enriched compound or a pharmaceutically acceptable salt thereof, R₁ is H, and each of R₂ and R₃ is deuterium enriched.

In yet another embodiment of the deuterium enriched compound or a pharmaceutically acceptable salt thereof, each of R₁, R₂ and R₃ is deuterium enriched.

In yet another embodiment of the deuterium enriched compound or a pharmaceutically acceptable salt thereof, the at least one of R₁-R₃ is deuterium enriched to have an isotopic purity of at least 10%.

In yet another embodiment of the deuterium enriched compound or a pharmaceutically acceptable salt thereof, the at least one of R₁-R₃ is deuterium enriched to have an isotopic purity of at least 50%.

In yet another embodiment of the deuterium enriched compound or a pharmaceutically acceptable salt thereof, the at least one of R₁-R₃ is deuterium enriched to have an isotopic purity of at least 70%.

In yet another embodiment of the deuterium enriched compound or a pharmaceutically acceptable salt thereof, the at least one of R₁-R₃ is deuterium enriched to have an isotopic purity of at least 90%.

In yet another embodiment of the deuterium enriched compound or a pharmaceutically acceptable salt thereof, the at least one of R₁-R₃ is deuterium enriched to have an isotopic purity of at least 95%.

In yet another embodiment of the deuterium enriched compound, the compound in the form of free base.

In yet another embodiment of the deuterium enriched compound, the compound is in the form of a pharmaceutically acceptable salt, wherein the pharmaceutically acceptable salt is selected from the group consisting of citrate, mesylate, maleate, malate, fumarate, tannate, tartrate, esylate, p-toluenesulfonate, benzoate, acetate, phosphate, oxalate and sulfate salts.

In yet another embodiment of the deuterium enriched compound, the compound is in the form of a mesylate salt or a citrate salt.

The subject invention also provides a pharmaceutical composition comprising the deuterium enriched compound described herein, or a pharmaceutically acceptable salt thereof, and a pharmaceutically acceptable carrier.

The subject invention further provides a mixture of at least two different deuterium enriched compounds, each compound having the structure:

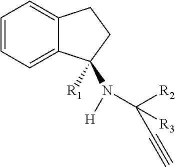

or pharmaceutically acceptable salts thereof, wherein R₁-R₃ are independently H or deuterium enriched.

In an embodiment of the mixture, at least one of the at least two deuterium enriched compounds has the structure:

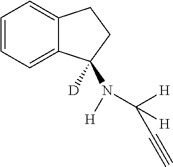

or pharmaceutically acceptable salts thereof.

In another embodiment of the mixture, at least one of the at least two deuterium enriched compounds has the structure:

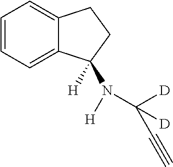

or pharmaceutically acceptable salts thereof.

In yet another embodiment of the mixture, at least one of the at least two deuterium enriched compounds has the structure:

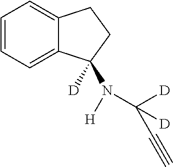

or pharmaceutically acceptable salts thereof.

The subject invention yet further provides a pharmaceutical composition comprising the mixture described herein, or a pharmaceutically acceptable salt thereof, and a pharmaceutically acceptable carrier.

The subject invention yet further provides a method of treating a neurodegenerative disorder in a subject in need thereof, the method comprising periodically administering to the subject in need a therapeutically effective amount of a dosage form comprising as an active ingredient the deuterium enriched compound described herein, or a pharmaceutically acceptable salt thereof, thereby to effectively treat the subject.

In an embodiment of the method, the therapeutically effective amount of the base form of the deuterium enriched compound is 0.2-2.5 mg per day.

In another embodiment of the method, the therapeutically effective amount of the base form of the deuterium enriched compound is 0.5 mg per day.

In yet another embodiment of the method, the therapeutically effective amount of the base form of the deuterium enriched compound is 1 mg per day.

In yet another embodiment of the method, the therapeutically effective amount of the base form of the deuterium enriched compound is 2 mg per day.

In yet another embodiment of the method, the dosage form is an oral dosage form.

In yet another embodiment of the method, the dosage form is a transdermal patch.

In yet another embodiment of the method, the neurodegenerative disorder is selected from the group consisting of Parkinson's disease, Restless Legs Syndrome, Multiple System Atrophy, Progressive Supranuclear Palsy, Glaucoma, Macular Degeneration, Hearing loss, Retinitis Pigmentosa, and Olfactory Dysfunction.

In yet another embodiment of the method, the neurodegenerative disorder is Parkinson's disease.

The subject invention yet further provides a method of reducing the rate of progression of Parkinson's disease in an early stage Parkinson's disease patient, the method comprising periodically administering to an early stage Parkinson's disease patient an amount of the deuterium enriched compound described herein, or a pharmaceutically acceptable salt thereof, effective to reduce the rate of progression of Parkinson's disease of the early stage Parkinson's disease patient.

The subject invention yet further provides a process for the preparation of a deuterium enriched compound having the structure:

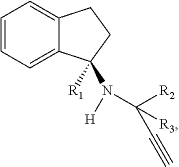

wherein R₁ is D and R₂ and R₃ are independently H or D, the process comprising:
d) reacting

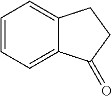

with LiAlD₄ in the presence of a solvent to obtain

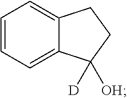

e) converting

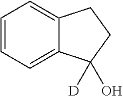

to obtain racemic N-propargyl aminoindan; and
f) separating the racemic N-propargyl aminoindan using a chiral separation method to obtain the compound.

In an embodiment of the process, in step a), the solvent is diethyl ether.

In another embodiment of the process, step b) comprises steps of:

i) reacting triethyl amine with 4-nitrobenzene-1-sulfonyl chloride in the presence of a first solvent to obtain

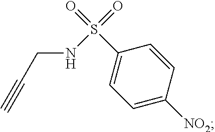

ii) reacting

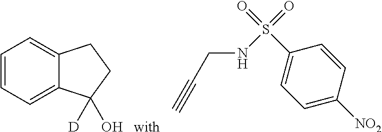

in the presence of a second solvent to obtain

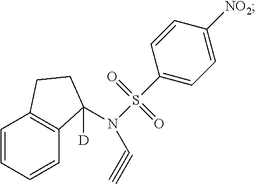

and
iii) reacting

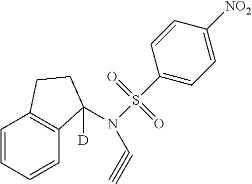

with an organic acid in the presence of a third solvent to obtain racemic N-propargyl aminoindan.

In an embodiment of the process, each of the first solvent and the second solvent is DCM and the third solvent is DMF.

In another embodiment of the process, in step iii) the organic is 2-mercaptoacetic acid.

In yet another embodiment of the process, step b) comprises steps of:

i) reacting

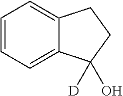

with diphenyl phosphorazedate and DBU in the presence of a first solvent to obtain

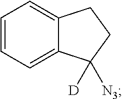

ii) reacting

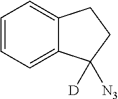

with hydrogen gas in the presence of a second solvent and a catalyst to obtain

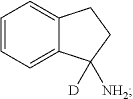

and
iii) reacting

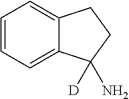

with DBU and

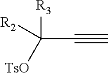

in the presence of a third solvent to obtain racemic N-propargyl aminoindan.

In yet another embodiment of the process, each of the first and third solvent is THF and the second solvent is MeOH.

In yet another embodiment of the process, in step ii) the catalyst is Pd/C.

In yet another embodiment of the process, the chiral separation method is SFC or chiral preparative HPLC in combination with SFC.

The subject invention yet further provides a process for the preparation of a compound having the structure:

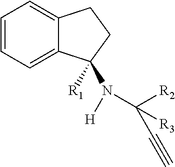

or a pharmaceutically acceptable salt thereof, wherein R₁ is H and R₂ and R₃ are independently H or D, and wherein at least one of R₂ and R₃ is deuterium enriched, the process comprising:

a) reacting methyl propiolate with LiAlD₄ in the presence of a first solvent to obtain

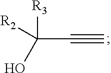

b) reacting

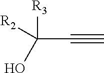

with TsCl and a base in the presence of a second solvent to obtain

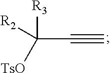

and
c) reacting

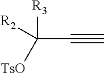

with (R)-1-aminoindan in the presence of a third solvent to obtain the compound.

In an embodiment of the process, in step b) the base is solid KOH.

In another embodiment of the process, each of the first and second solvent is ethyl ether and the third solvent is THF.

Deuterium (D or 2H) is a stable, non-radioactive isotope of hydrogen and has an atomic weight of 2.0144. Hydrogen atom in a compound naturally occurs as a mixture of the isotopes 1H (hydrogen or protium), D (2H or deuterium), and T (3H or tritium). The natural abundance of deuterium is 0.0156%. Thus, a compound with a level of deuterium at any site of hydrogen atom in the compound that has been enriched to be greater than its natural abundance of 0.0156%, is novel over its non-enriched counterpart.

As used herein, a “deuterium-enriched” compound means that the abundance of deuterium at any relevant site of the compound is more than the abundance of deuterium naturally occurring at that site in an amount of the compound. A relevant site in a compound as used above is a site which would be designated as “H” in a chemical structure representation of the compound when not deuterium-enriched. Naturally occurring as used above refers to the abundance of deuterium which would be present at a relevant site in a compound if the compound was prepared without any affirmative step to enrich the abundance of deuterium. Thus, in a “deuterium-enriched” compound, the abundance of deuterium at any of its relevant sites can range from more than 0.0156% to 100%. Examples of ways to obtain a deuterium-enriched compound are exchanging hydrogen with deuterium or synthesizing the compound with deuterium-enriched starting materials.

Obtaining 100% deuteration at any relevant site of a compound in an amount of milligram or greater can be difficult. Therefore, it is understood that some percentage of hydrogen may still be present, even though a deuterium atom is specifically shown in a chemical structure. Thus, when a chemical structure contains a “D”, the compound represented by the structure is deuterium-enriched at the site represented by “D”.

A characteristic of a compound refers to any quality that a compound exhibits, e.g., peaks or retention times, as determined by 1H nuclear magnetic spectroscopy, mass spectroscopy, infrared, ultraviolet or fluorescence spectrophotometry, gas chromatography, thin layer chromatography, high performance liquid chromatography, elemental analysis, Ames test, dissolution, stability and any other quality that can be determined by an analytical method. Once the characteristics of a compound are known, the information can be used to, for example, screen or test for the presence of the compound in a sample.

As used herein, a “pharmaceutically acceptable” carrier or excipient is one that is suitable for use with humans and/or animals without undue adverse side effects (such as toxicity, irritation, and allergic response) commensurate with a reasonable benefit/risk ratio.

A “pharmaceutically acceptable salt” of rasagiline, as well as of the deuterated compounds herein, includes citrate, mesylate, maleate, malate, fumarate, tannate, tartrate, esylate, p-toluenesulfonate, benzoate, acetate, phosphate, oxalate and sulfate salts. For the preparation of pharmaceutically acceptable acid addition salts of the compounds of the invention, the free base can be reacted with the desired acids in the presence of a suitable solvent by conventional methods.

As used herein, “drug substance” refers to the active ingredient in a drug product, which provides pharmacological activity or other direct effect in the diagnosis, cure, mitigation, treatment, or prevention of disease, or to affect the structure or any function of the body of man or animals.

As used herein, “drug product” refers to the finished dosage form containing the drug substance as well as at least one pharmaceutically acceptable carrier.

As used herein, an “isolated” compound is a compound separated by an affirmative act from the crude reaction mixture in which the compound was first formed. The separation of the compound is from the other known components of the crude reaction mixture, with some impurities, unknown side products and residual amounts of the other known components of the crude reaction mixture permitted to remain. Purification is an example of an affirmative act of isolation.

As used herein, a composition that is “free” of a chemical entity means that the composition contains, if at all, an amount of the chemical entity which cannot be avoided following an affirmative act intended to eliminate the presence of chemical entity in the composition.

As used herein, “stability testing” refers to tests conducted at specific time intervals and various environmental conditions (e.g., temperature and humidity) to see if and to what extent a drug product degrades over its designated shelf life time. The specific conditions and time of the tests are such that they accelerate the conditions the drug product is expected to encounter over its shelf life. For example, detailed requirements of stability testing for finished pharmaceuticals are codified in 21 C.F.R §211.166, the entire content of which is hereby incorporated by reference.

As used herein, a “neurodegenerative disorder” is a disorder in which progressive loss of neurons occurs either in the peripheral nervous system or in the central nervous system. Non-limiting examples of neurodegenerative disorders include Parkinson's disease, Restless Legs Syndrome, Multiple System Atrophy (MSA), Progressive Supranuclear Palsy (PSP), Glaucoma, Macular Degeneration, Hearing loss, Retinitis Pigmentosa, and Olfactory Dysfunction.

As used herein, “about” in the context of a numerical value or range means±10% of the numerical value or range recited or claimed.

A dosage unit may comprise a single compound or mixtures of compounds thereof. A dosage unit can be prepared for oral dosage forms, such as tablets, capsules, pills, powders, and granules.

R(+)PAI or deuterated compounds (deuterated R(+)PAI) disclosed herein may be obtained by optical resolution of racemic mixtures of R and S-enantiomer of N-propargyl-1-aminoindan (PAI). Such a resolution can be accomplished by any conventional resolution method, well known to a person skilled in the art, such as those described in “Enantiomers, Racemates and Resolutions” by J. Jacques, A. Collet and S. Wilen, Pub. John Wiley & Sons, N.Y., 1981. For example, the resolution may be carried out by preparative chromatography on a chiral column. Another example of a suitable resolution method is the formation of diastereomeric salts with a chiral acid such as tartaric, malic, mandelic acid or N-acetyl derivatives of amino acids, such as N-acetyl leucine, followed by recrystallisation to isolate the diastereomeric salt of the desired R enantiomer.

The racemic mixture of R and S enantiomers of PAI may be prepared, e.g. as described in WO 95/11016. The racemic mixture of PAI can also be prepared by reacting 1-chloroindan or 1-bromoindan with propargylamine. Alternatively, this racemate may be prepared by reacting propargylamine with 1-indanone to form the corresponding imine, followed by reduction of the carbon-nitrogen double bond of the imine with a suitable agent, such as sodium borohydride.

Rasagiline or deuterated compounds disclosed herein may be prepared as pharmaceutical compositions particularly useful for treating: Parkinson's disease, brain ischemia, head trauma injury, spinal trauma injury, neurotrauma, neurodegenerative disease, neurotoxic injury, nerve damage, dementia, Alzheimer's type dementia, senile dementia, depression, memory disorders, hyperactive syndrome, attention deficit disorder, multiple sclerosis, schizophrenia, and/or affective illness, but with a reduced risk of peripheral MAO inhibition that is typically associated with administration of rasagiline with known oral dosage forms.

Specific examples of pharmaceutically acceptable carriers and excipients that may be used to formulate oral dosage forms of the present invention are described, e.g., in U.S. Pat. No. 6,126,968 to Peskin et al., issued Oct. 3, 2000. Techniques and compositions for making dosage forms useful in the present invention are described, for example, in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

Tablets may contain suitable binders, lubricants, disintegrating agents, coloring agents, flavoring agents, flow-inducing agents, melting agents, stabilizing agents, solubilizing agents, antioxidants, buffering agent, chelating agents, fillers and plasticizers. For instance, for oral administration in the dosage unit form of a tablet or capsule, the active drug component can be combined with an oral, non-toxic, pharmaceutically acceptable, inert carrier such as gelatin, agar, starch, methyl cellulose, dicalcium phosphate, calcium sulfate, mannitol, sorbitol, microcrystalline cellulose and the like. Suitable binders include starch, gelatin, natural sugars such as corn starch, natural and synthetic gums such as acacia, tragacanth, or sodium alginate, povidone, carboxymethylcellulose, polyethylene glycol, waxes, and the like. Antioxidants include ascorbic acid, fumaric acid, citric acid, malic acid, gallic acid and its salts and esters, butylated hydroxyanisole, editic acid. Lubricants used in these dosage forms include sodium oleate, sodium stearate, sodium benzoate, sodium acetate, stearic acid, sodium stearyl fumarate, talc and the like. Disintegrators include, without limitation, starch, methyl cellulose, agar, bentonite, xanthan gum, croscarmellose sodium, sodium starch glycolate and the like, suitable plasticizers include triacetin, triethyl citrate, dibutyl sebacate, polyethylene glycol and the like.

The compositions may be prepared as medicaments to be administered orally, parenterally, rectally or transdermally. Suitable forms for oral administration include tablets, compressed or coated pills, dragees, sachets, hard or soft gelatin capsules, sublingual tablets, syrups and suspensions; for parenteral administration the invention provides ampoules or vials that include an aqueous or non-aqueous solution or emulsion; for rectal administration there are provided suppositories with hydrophilic or hydrophobic vehicles; and for topical application as ointments and transdermal delivery there are provided suitable delivery systems as known in the art.

Transdermal Formulations are medicated adhesive patches placed on the skin to deliver a time-released dose of medication through the skin and into the bloodstream. A wide variety of pharmaceuticals can be delivered through transdermal patches, such as nicotine for smoking cessation, scopolamine for motion sickness, estrogen for menopause, and prevention of osteoporosis, nitroglycerin for angina, lidocaine for pain relief from shingles. Some pharmaceuticals must be combined with other substances, such as alcohol, to increase their ability to penetrate the skin. Molecules of insulin, and many other pharmaceuticals, however, are too large to pass through the skin. Transdermal patches have several important components, including a liner to protect the patch during storage, the drug, adhesive, a membrane (to control release of the drug from the reservoir), and a backing to protect the patch from the outer environment. The two most common types of transdermal patches are matrix and reservoir types. (“Transdermal Patches” Wikipedia, Nov. 15, 2007, Wikipedia Foundation, Inc., Dec. 13, 2007 en.wikipedia.org/wiki/Transdermal patch; and Remington, The Science and Practice of Pharmacy, 20^{th} Edition, 2000).

In reservoir type patches, a drug is combined with a non-volatile, insert liquid, such as mineral oil, whereas drug in matrix type patches a drug is dispersed in a lipophilic or hydrophilic polymer matrix such as acrylic or vinylic polymers. Adhesive polymers, such as polyisobutylene, are used to hold the patch in place on the skin. (Stanley Scheindlin, (2004) “Transdermal Drug Delivery: PAST PRESENT, FUTURE,” Molecular Interventions, 4:308-312).

The major limitation to transdermal drug-delivery is the intrinsic barrier property of the skin. Penetration enhancers are often added to transdermal drug formulations in order to disrupt the skin surface and cause faster drug delivery. Typical penetration enhancers include high-boiling alcohols, diols, fatty acid esters, oleic acid and glyceride-based solvents, and are commonly added at a concentration of one to 20 percent (w/w). (Melinda Hopp, “Developing Custom Adhesive Systems for Transdermal Drug Delivery Products,” Pharmaceutical Technology, March 2002, pages 30-36).

Rasagiline or the deuterated compounds disclosed herein, may be used alone, or alternatively, they may be used as an adjunct to existing treatments. The disclosed compounds may be administered at different times and separate from other treatments, or as a combined pharmaceutical composition with other treatments. Thus, for example, a pharmaceutical composition for oral use in the form of tablets or capsules may comprise a disclosed compound, Levodopa, and a decarboxylase inhibitor. Such a composition may comprise 0.01-20 mg of a disclosed compound in base form, 50-100 mg of Levodopa, and 12.5-50 mg of benserazide.

The preferred dosages of R(+)PAI or its deuterated forms in any of the disclosed compositions may be within the following ranges: for oral or suppository formulations 0.01-20 mg per dosage unit to be taken daily, preferably 0.5-5 mg per dosage unit to be taken daily and more preferably 1 mg or 2 mg per dosage unit to be taken daily may be used; and for injectable formulations 0.05-10 mg/ml per dosage unit to be taken daily and more preferably 0.5-3 mg/ml per dosage unit to be taken daily and more preferably 1 mg/ml per dosage unit to be taken daily may be used. The amounts herein refer to the weight of the base compound, not the salt form thereof.

By any range disclosed herein, it is meant that all hundredth, tenth and integer unit amounts within the range are specifically disclosed as part of the invention. Thus, for example, 0.01 mg to 50 mg means that 0.02, 0.03 . . . 0.09; 0.1, 0.2 . . . 0.9; and 1, 2 . . . 49 mg unit amounts are included as embodiments of this invention. For example, a range of 0.01-20 mg means that all hundredth, tenth and integer unit amounts within the range are specifically disclosed as part of the invention. Thus, 0.02, 0.03 . . . 0.09; 0.1, 0.2 . . . 0.9; and 1, 2 . . . 19 mg unit amounts are included as embodiments of this invention.

Metabolites from chemical compounds, whether inherent or pharmaceutical, are formed as part of the natural biochemical process of degrading and eliminating the compounds. The rate of degradation of a compound is an important determinant of the duration and intensity of its action. Profiling metabolites of pharmaceutical compounds, drug metabolism, is an important part of drug discovery, leading to an understanding of any undesirable side effects.

### Metabolization of Rasagiline

Rasagiline is slowly metabolized by CYP1A2 to form several primary metabolites as shown below:

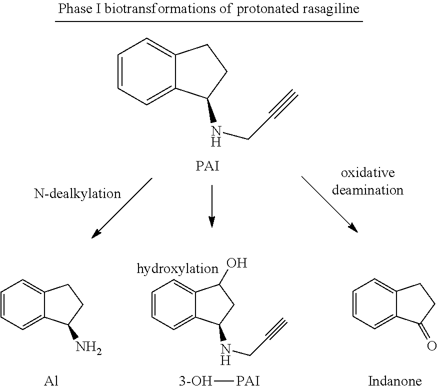

These primary metabolites may undergo further metabolism by Phase 1 or 2 metabolic reactions.

Drugs deuterated (C-D instead of C-H) at the site of metabolic biotransformation are more resistant to metabolic changes, especially when those changes are mediated by cytochrome P450 systems. This is due to so called Deuterium Kinetic Isotope Effect (DKIE). Thus, deuterated forms of rasagiline may have different metabolic profile than the protonated forms. The increased levels of deuterium incorporation may produce a detectable DKIE that could affect the pharmacokinetic, pharmacologic and/or toxicologic profiles of rasagiline compared with rasagiline having naturally occurring levels of deuterium.

Deuteration of the C—H bond to be oxidized may also change the pathway of drug metabolism (metabolic switching). The metabolic scheme below illustrates different ways to slow CYP1A2-mediated metabolism of rasagiline, such as

a) des propargylation:

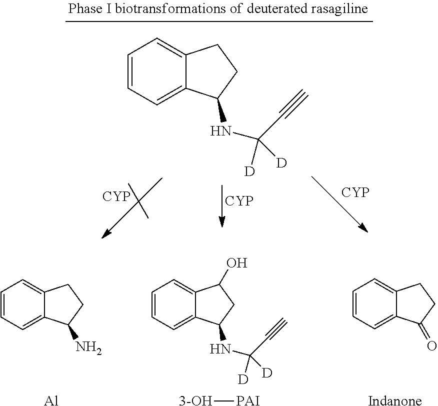

b) hydroxylation:

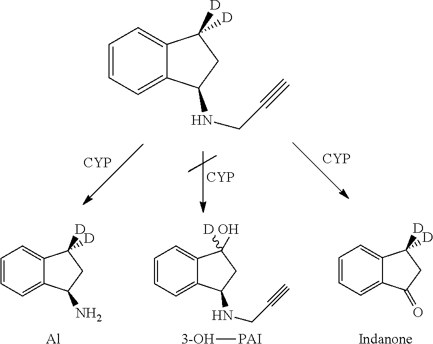

c) oxidative deamination:

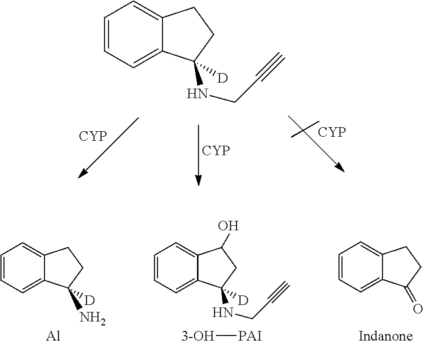

Literature illustrates a similar pathway blocking in the phenalzine molecule, which is also potent MAO inhibitor but catabolized by MAO. (Dyck LE et al., “Effect of chronic deuterated and non-deuterated phenelzine on rat brain monoamines and monoamine oxidase”, Naunyn Schmiedebergs Arch Pharmacol., 1988 March; 337(3):279-83). The phenalzine molecule studied in Dyck LE et al. was modified by deuteration at alpha and beta carbons of hydrazine moiety as shown below:

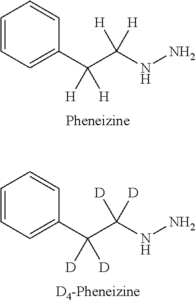

Study results in Dyck LE et al. indicated that the deuterated phenelzine is a more potent MAO inhibitor, but not in-vitro meaning most probably higher stability to oxidation. Another potential mechanism discussed in Dyck LE et al. was an increased neuronal uptake which was also shown for other amines, e.g. D3-NA.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

Certain reagents to be used in the following examples are listed in the table below.

| Reagents | CAS No. |
|---|---|
| 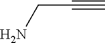 | 2450-71-7 |
| 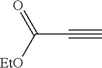 | 623-47-2 |
| 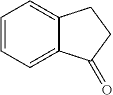 | 83-33-0 |
| LiAlD4 (EM 42) | 14128-54-2 |
| 98 atom % D, 90% (CP) | |
| Lithium aluminum deuteride solution | 14128-54-2 |
| (1.0 M in diethyl ether, 96 atom % D) | |
| Lithium aluminum deuteride | 14128-54-2 |
| solution 1.0 M in THF, 96 atom % D | |

### Example 1

### Synthesis of Compound 1

Compound 1 was prepared via a synthetic route described in the following reaction scheme:

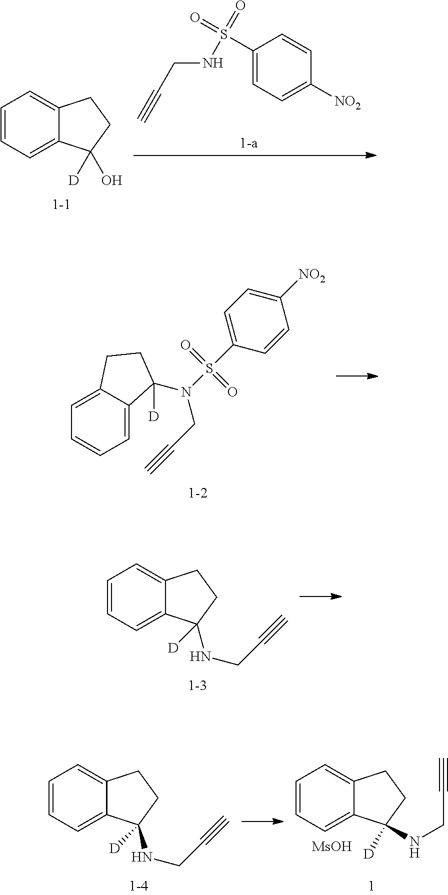

### Step 1

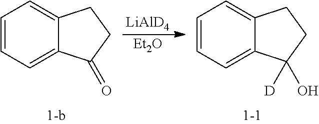

To a suspension of LiAlD₄ (5 g) in 100 ml diethyl ether cooled to −70° C. was added dropwise a solution of Compound 1-b (30 g) in diethyl ether (100 ml). The addition was completed in 2 hours. After addition, the reaction mixture was stirred at −70° C. for another 2 hours. Aqueous NaOH (5 g, 15% solution) and 15 g of water were then added. The precipitate was filtered and washed with ether. The combined ethereal fractions were concentrated in vacuum to provide Compound 1-1 as clear oil (27 g, 80%).

### Step 2

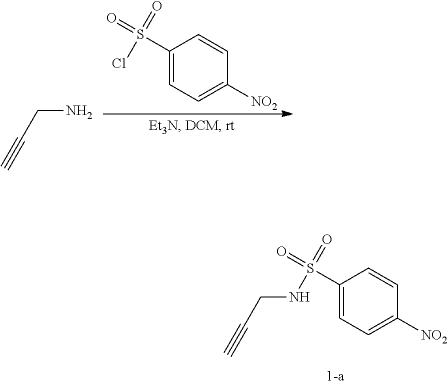

To a solution of prop-2-yn-1-amine (11 g) in 500 ml of Dichloromethane (DCM) were added 41 g of triethyl amine and 44 g of 4-nitrobenzene-1-sulfonyl chloride. The reaction mixture was stirred at room temperature overnight. The resulting solution was added 500 ml saturated NaHCO₃ solution while stirring. During addition precipitation was taken place, the precipitate was filtered, washed with water several times and dried to provide Compound 1-a in 75% yield (36 g).

### Step 3

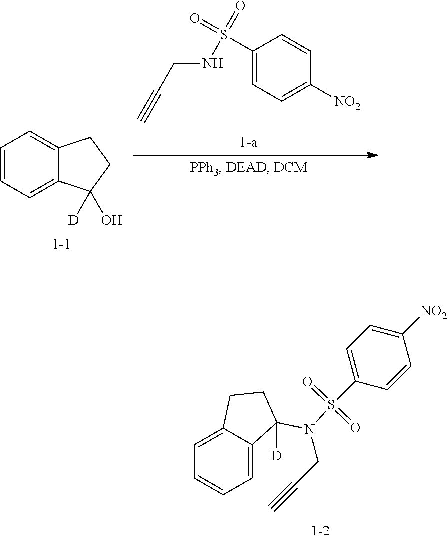

A solution of Compound 1-1 (2.7 g, 0.02 mol), Compound 1-a (4.8 g, 0.02 mol) and triphenylphosphine (11 g, 0.042 mol) in 200 ml DCM was cooled to 0° C. and diethyl azodicarboxylate (DEAD) (7.3 g 0.042 mol) was added. The resulting mixture was warmed to room temperature and stirred overnight. After the reaction was completed, the reaction mixture was concentrated and was further purified by flash chromatography using 35% of ethyl acetate (EA) in petrol ether (PE) to give 3.6 g of Compound 1-2 in 50% yield.

### Step 4

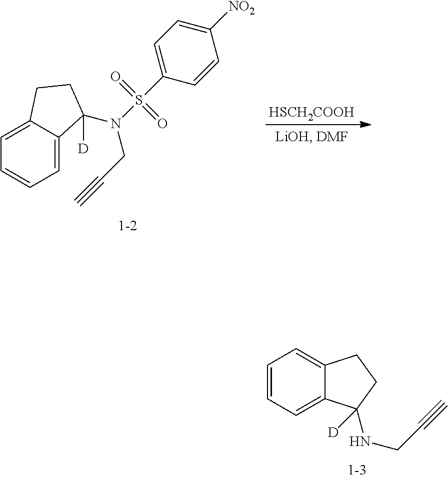

To a stirred solution of Compound 1-2 (1.8 g, 5 mmol) in DMF (50 mL) were added 2-mercaptoacetic acid (0.91 g, 10 mmol) and LiOH (0.48 g, 20 mmol). The resulting mixture was stirred overnight and partitioned between 200 ml ether and 100 ml saturated NaHCO₃ solution. The ether phase was collected, concentrated and was further purified by flash chromatography using 20% gradient of EA in PE to give 0.77 g of Compound 1-3 in 90% yield.

### Step 5

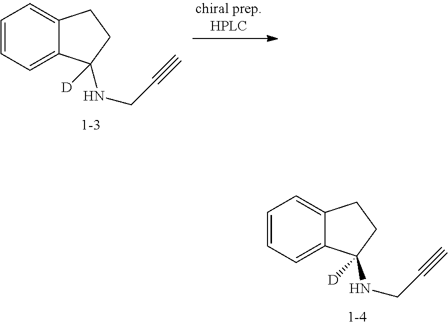

Deuterated R-rasagiline free base (Compound 1-4) was obtained following 2 times chiral preparation using Supercritical Fluid Chromatography (SFC).

### Step 6

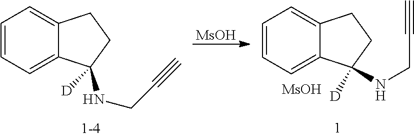

Deuterated R-rasagiline free base (Compound 1-4) (1.1 g) was dissolved in 8 g of isopropanol and 0.7 g of methanesulfonic acid was added at stirring and cooling. During the addition crystallization of rasagiline mesylate took place. The resulting suspension was heated to reflux and after complete dissolution of solids was cooled to 10° C. At cooling rasagiline mesylate crystallized, the mixture was stirred at 10° C. for 15 minutes and then filtered. The solid product was washed with ether and was dried under vacuum at 60° C. to obtain Compound 1. Compound 1 elutes at good chromatographic purity (99.2% area in a HPLC chromatogram).

H-NMR (CD₃OD): δ=7.57-7.59 (d, 1H), 7.40-7.43 (m, 2H), 7.35-7.37 (m, 1H), 4.0 (s, 2H), 3.3 (s, 1H), 3.19-3.24 (m, 1H), 3.03-3.06 (m, 1H), 2.7 (s, 3H), 2.58-2.61 (m, 1H), 2.26-2.32 (m, 1H).

### Example 2

### Synthesis of Compound 2

Compound 2 was prepared via a synthetic route described in the following reaction scheme:

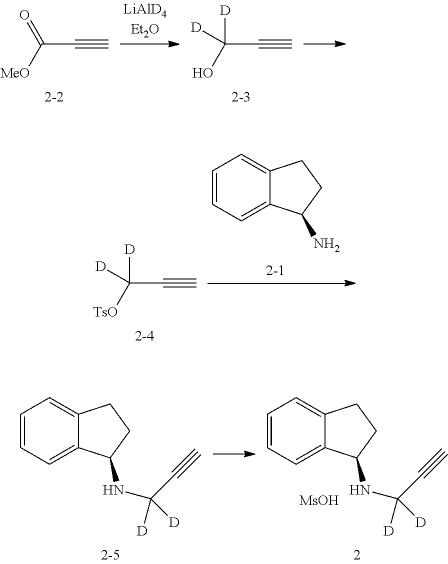

### Step 1

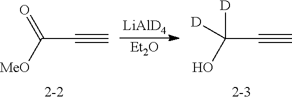

Lithium aluminum deuteride (5.0 g, 0.12 mol) was suspended in 200 mL of ethyl ether in a 500-mL round-bottom flask fitted with an addition funnel. The flask was cooled to −78° C., methyl propiolate (13.5 g, 0.16 mol) in 100 mL of ether was added drop wise over 4 h. After complete addition, the solution was stirred at −40° C. overnight. Water (5.0 g) was added drop wise to the flask, which was then allowed to warm to room temperature. Aqueous NaOH (5 g, 15% solution) and 15 g of water were then added. The precipitate was filtered and washed with ether. The combined ethereal fractions were evaporated and distilled at atmospheric pressure. The residue containing prop-2-yn-1-ol (Compound 2-3) was used directly for the next step.

### Step 2

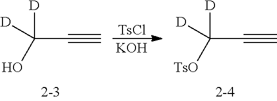

The residue containing Compound 2-3 (containing ˜0.16 mol of Compound 2-3) and 4-toluenesulfonyl chloride (TsCl) (60 g, 0.32 mol) in 500 ml ether was cooled to −10° C. To this solution was added KOH solid (90 g, 1.6 mol). After addition the reaction mixture was warmed to room temperature and stirred for 2 hours. The reaction mixture was filtered and the filtrate was concentrated in vacuum to provide Compound 2-4 as a yellow residue which was used directly for the next step.

### Step 3

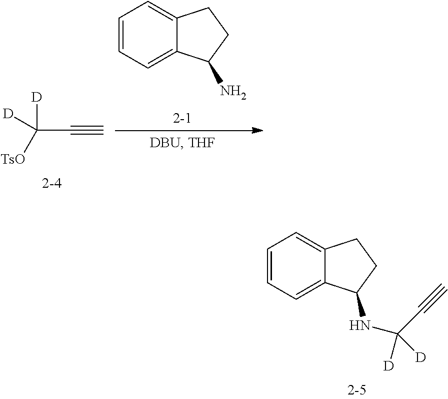

(R)-1-Aminoindan (1.33 g, 10 mmol 1 eq) was dissolved in dry THF (20 ml) and cooled to 0-5° C. 1,8-Diazabicyclo[5.4.0]undec-7-ene (DBU) (1.5 equivalents) was added slowly and the resultant mixture stirred for 30 minutes before propargyl tosylate (Compound 2-4 from Step 2, 1.25 equivalents) was added dropwise. The reaction mixture was stirred for 4 hours at 15-20° C. After completion of the reaction, the THF was removed under reduced pressure and water (30 ml) added. The product was extracted with DCM (3×50 ml), followed by washing with 10% aqueous NaOH (2×30 ml) and water (2×3 ml). The DCM was removed under reduced pressure at 40° C. to obtain a residue. The residue was then purified by preparative HPLC to provide Compound 2-5 as colorless oil.

### Step 4

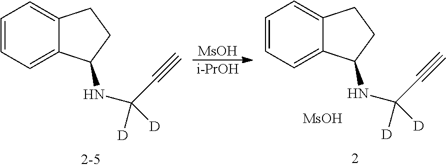

Deuterated rasagiline base (Compound 2-5) (1.1 g) was dissolved in 8 g of isopropanol and 0.7 g of methanesulfonic acid was added at stirring and cooling. During the addition crystallization of rasagiline mesylate took place. The resulting suspension was heated to reflux and cooled to 10° C. after complete dissolution of solid. At cooling rasagiline mesylate crystallized, the mixture stirred at 10° C. for 15 minutes and filtered. Solid product was washed with ether and dried in vacuum at 60° C. to provide Compound 2. Compound 2 elutes at good chromatographic purity (98.5% area in a HPLC chromatogram).

### ¹H-NMR (D₂O): δ=7.36-7.58 (m, 4H), 4.94-4.94 (q, 1H), 3.03-3.24 (m, 3H), 3.19-3.24 (m, 1H), 2.8 (s, 3H), 2.50-2.63 (m, 1H), 2.25-2.35 (m, 1H).

### Example 3

### Synthesis of Compound 3

Compound 3 was prepared via a synthetic route described in the following reaction scheme:

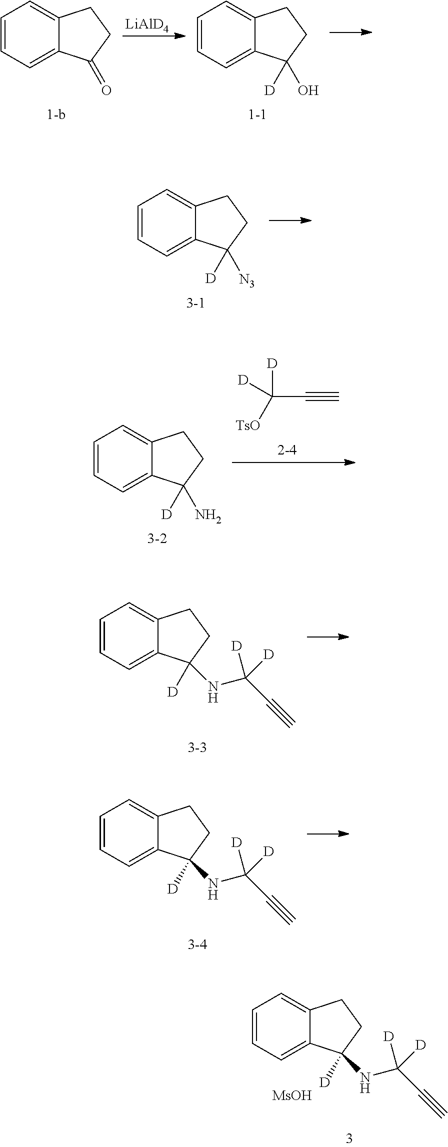

### Step 1

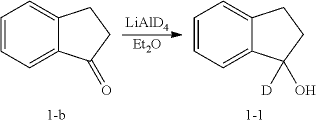

To a suspension of LiAlD₄ (5 g) in 100 ml diethyl ether cooled to −70° C. was added dropwise a solution of Compound 1-b (30 g) in diethyl ether (100 ml). The addition was completed in 2 hours. After addition, the reaction mixture was stirred at −70° C. for another 2 hours. Aqueous NaOH (5 g, 15% solution) and 15 g of water were then added. The precipitate was filtered and washed with ether. The combined ethereal fractions were concentrated in vacuum to provide Compound 1-1 as clear oil (27 g, 80%).

### Step 2

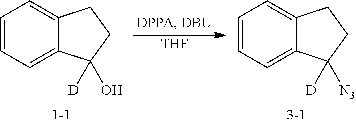

A mixture of alcohol Compound 1-1 (13.5 g, 0.1 mol) and diphenyl phosphorazidate (30 g, 0.11) was dissolved in dry THF (100 mL). The mixture was cooled to 0° C. under N₂, and neat DBU (20 g, 0.13 mol) was added. The mixture was stirred at room temperature overnight. The resulting mixture was partitioned between DCM (500 ml) and water (400 ml). The organic phase was separated, washed with water (200 mL) and 5% HCl (200 mL). The organic layer was concentrated in vacuum and the residue was purified by silica gel chromatography using 95:5 hexane/ethyl acetate to afford 12 g (75%) Compound 3-1 as clear, colorless oil.

### Step 3

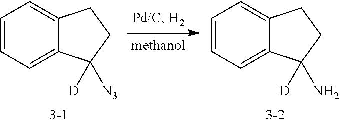

To a solution of Compound 3-1 (3.2 g, 0.02 mol) in 100 mL of MeOH was added 0.5 g of 10% Pd/C. The mixture was stirred under H₂ atmosphere overnight. After the reaction was completed, the mixture was filtered through Celite and the filtrate was evaporated in vacuum. Compound 3-2 was obtained as a light yellow liquid (2.4 g, 90%).

### Step 4

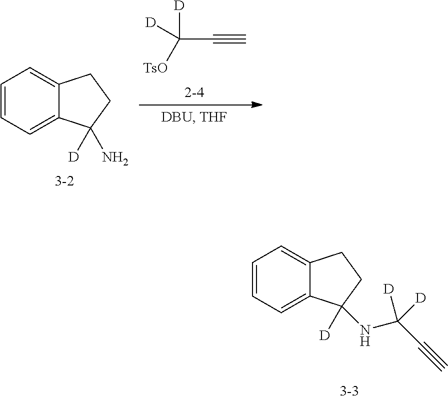

Compound 3-2 (1.34 g, 10 mmol, 1 eq.) was dissolved in dry THF (20 ml) and cooled to 0-5° C. DBU (1.5 equivalents) was added slowly and the resultant mixture stirred for 30 minutes before propargyl tosylate (Compound 2-4 prepared according to Step 2 of Example 2) (1.25 equivalents) was added dropwise. The reaction mixture was stirred for 4 hours at 15-20° C. After completion of the reaction, the THF was removed under reduced pressure and water (30 ml) added. The product was extracted with DCM (3×50 ml), followed by washing with 10% aqueous NaOH (2×30 ml) and water (2×3 ml). The DCM was removed under vacuum to obtain a residue which was purified by preparative HPLC to provide Compound 3-3 as clear oil.

### Step 5

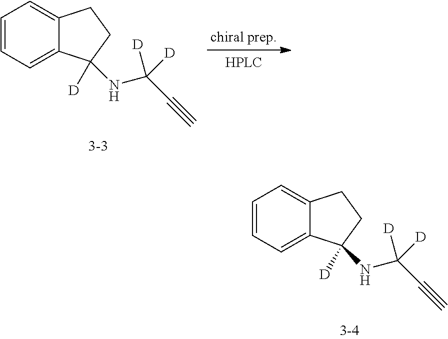

Deuterated R-rasagiline free base (Compound 3-4) was obtained after 3 times chiral preparative HPLC and 2 times SFC.

### Step 6

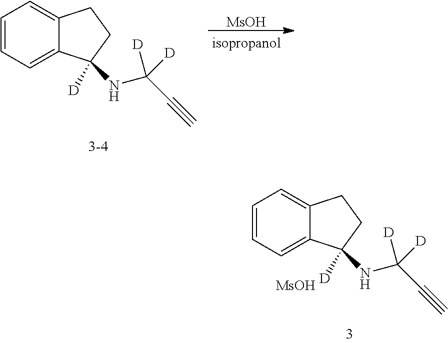

Deuterated R-rasagiline free base (Compound 3-4) (1.33 g) was dissolved in 8 g of isopropanol and 0.8 g of methanesulfonic acid was added at stirring and cooling. During the addition crystallization of rasagiline mesylate took place. The resulting suspension was heated at stirring to reflux and then cooled to 10° C. after complete dissolution of solid. At cooling rasagiline mesylate was crystallized, the mixture stirred at 10° C. for 15 minutes and filtered. Solid product was washed with ether and dried under vacuum at 60° C. to provide Compound 3. Compound 3 elutes at good chromatographic purity (99.0% area in a HPLC chromatogram).

¹H-NMR (CD₃OD): δ=7.5 (d, 1H), 7.40-7.45 (m, 2H), 7.33-7.37 (m, 1H), 3.3 (s, 1H), 3.2 (m, 1H), 3.0 (m, 1H), 2.7 (s, 3H), 2.5-2.7 (m, 1H), 2.2-2.3 (m, 1H).

### Example 4

### Pharmacokinetic and Partial Metabolic Evaluation

Compounds 1, 2, and 3 used in this example are prepared according to the processes described in Examples 1, 2, and 3, respectively, and rasagline used is manufactured by Teva Pharmaceuticals Industries Ltd. (Petach Tikva, Israel).

Test formulations containing Compounds 1, 2, 3, or rasagiline at 1 mg dose level are prepared and are administered to mice. A “dose level”, such as in “1 mg dose level”, refers to the weight of the base form of Compounds 1, 2, 3, or rasagiline, not the weight of the corresponding salt form, which is heavier. The concentrations of Compound 1, Compound 2, Compound 3, and rasagiline in the plasma samples of the mice are determined using reliable LC/MS/MS assay. Pharmacokinetic parameters are calculated and the mean plasma levels versus time curves are evaluated.

The following pharmacokinetic parameters are determined from the mean plasma concentration-time data (mean of three animals at each time point) of Compound 1, Compound 2, Compound 3 and rasagiline.

| Parameter | Definition |
|---|---|
| AUC(0-t) | Area under the plasma concentration-time curve |
| | from time zero up to time of last detectable |
| | concentration (tz) |
| AUC(0-∞) | Area under the plasma concentration-time curve |
| | from time zero up to infinity |
| Cₘₐₓ | Maximum observed plasma concentration |
| tₘₐₓ | Time of maximum observed plasma concentration |
| t_{1/2} | Apparent terminal elimination half-life |

The testing results of this example show that the average plasma concentration of deuterium-enriched rasagiline is comparable to that of non-deuterated rasagiline.

The testing results of this example also show that deuterium-enriched rasagiline reduces the formation of metabolites, while maintaining a similar plasma concentration-time profile to that of non-deuterated rasagiline.

### Example 5

### Assessment of Efficacy of Deuterated Rasagiline (Compounds 1, 2 and 3)

Rasagiline has been shown to be active against various diseases in various models, for example, as described in U.S. Pat. No. 5,387,612.

In this example, Compounds 1, 2, and 3 used are prepared according to the processes described in Examples 1, 2, and 3, respectively. Each of Compounds 1, 2 and 3 are individually tested using the models as described in U.S. Pat. No. 5,387,612 and are each found to have similar activity when compared to the activity of rasagiline which is not deuterium enriched.

Based on the similarity of activity with rasagiline, dosing parameters for deuterium-enriched rasagiline are developed.

1. A deuterium enriched compound having the structure: 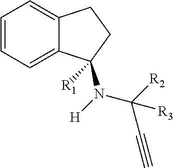
or a pharmaceutically acceptable salt thereof,
wherein R₁-R₃ are independently H or D, and wherein at least one of R₁-R₃ is deuterium enriched.

2. The deuterium enriched compound of claim 1 or a pharmaceutically acceptable salt thereof, wherein R₁ is deuterium enriched, and each of R₂ and R₃ is H.

3. The deuterium enriched compound of claim 1 or a pharmaceutically acceptable salt thereof, wherein R₁ is H, and each of R₂ and R₃ is deuterium enriched.

4. The deuterium enriched compound of claim 1 or a pharmaceutically acceptable salt thereof, wherein each of R₁, R₂ and R₃ is deuterium enriched.

5. The deuterium enriched compound of claim 1 or a pharmaceutically acceptable salt thereof, wherein the at least one of R₁-R₃ is deuterium enriched to have an isotopic purity of at least 10%.

6. The deuterium enriched compound of claim 1 or a pharmaceutically acceptable salt thereof, wherein the at least one of R₁-R₃ is deuterium enriched to have an isotopic purity of at least 50%.

7. The deuterium enriched compound of claim 1 or a pharmaceutically acceptable salt thereof, wherein the at least one of R₁-R₃ is deuterium enriched to have an isotopic purity of at least 70%.

8. The deuterium enriched compound of claim 1 or a pharmaceutically acceptable salt thereof, wherein the at least one of R₁-R₃ is deuterium enriched to have an isotopic purity of at least 90%.

9. The deuterium enriched compound of claim 1 or a pharmaceutically acceptable salt thereof, wherein the at least one of R₁-R₃ is deuterium enriched to have an isotopic purity of at least 95%.

10. The deuterium enriched compound of claim 1, in the form of free base.

11. The deuterium enriched compound of claim 1, in the form of a pharmaceutically acceptable salt, wherein the pharmaceutically acceptable salt is selected from the group consisting of citrate, mesylate, maleate, malate, fumarate, tannate, tartrate, esylate, p-toluenesulfonate, benzoate, acetate, phosphate, oxalate and sulfate salts.

12. The deuterium enriched compound of claim 11, in the form of a mesylate salt or a citrate salt.

13. A pharmaceutical composition comprising the deuterium enriched compound of claim 1, or a pharmaceutically acceptable salt thereof, and a pharmaceutically acceptable carrier.

14. A mixture of at least two different deuterium enriched compounds, each compound having the structure: 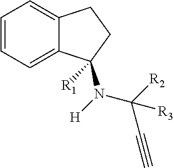
or pharmaceutically acceptable salts thereof, wherein R₁-R₃ are independently H or deuterium enriched.

15.-17. (canceled)

18. A pharmaceutical composition comprising the mixture of claim 14, or a pharmaceutically acceptable salt thereof, and a pharmaceutically acceptable carrier.

19. A method of treating a neurodegenerative disorder in a subject in need thereof, the method comprising periodically administering to the subject in need a therapeutically effective amount of a dosage form comprising as an active ingredient the deuterium enriched compound of claim 1, or a pharmaceutically acceptable salt thereof, thereby to effectively treat the subject.

20. The method of claim 19, wherein the therapeutically effective amount of the base form of the deuterium enriched compound is 0.2-2.5 mg per day.

21.-27. (canceled)

28. A method of reducing the rate of progression of Parkinson's disease in an early stage Parkinson's disease patient, the method comprising periodically administering to an early stage Parkinson's disease patient an amount of the deuterium enriched compound of claim 1, or a pharmaceutically acceptable salt thereof, effective to reduce the rate of progression of Parkinson's disease of the early stage Parkinson's disease patient.

29. A process for the preparation of a deuterium enriched compound having the structure: 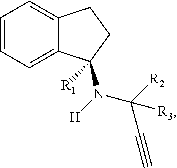
wherein R₁ is D and R₂ and R₂ are independently H or D, the process comprising:
a) reacting 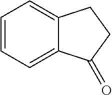
with LiAlD₄ in the presence of a solvent to obtain 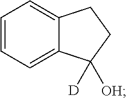
b) converting 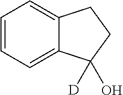
to obtain racemic N-propargyl aminoindan; and
c) separating the racemic N-propargyl aminoindan using a chiral separation method to obtain the compound.

30.-37. (canceled)

38. A process for the preparation of a compound having the structure: 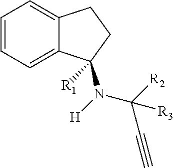
or a pharmaceutically acceptable salt thereof, wherein R₁ is H and R₂ and R₃ are independently H or D, and wherein at least one of R₂ and R₃ is deuterium enriched,
the process comprising:
a) reacting methyl propiolate with LiAlD₄ in the presence of a first solvent to obtain 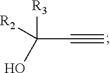
b) reacting 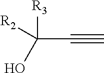
with TsCl and a base in the presence of a second solvent to obtain 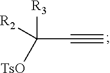
and
c) reacting 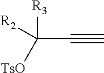
with (R)-1-aminoindan in the presence of a third solvent to obtain the compound.

39.-40. (canceled)
